(19) United States
(12) Patent Application Publication
(10) Pub. No.: US 2002/0076382 A1
(43) Pub. Date: Jun. 20, 2002

(54) Formulations of mometasone and a bronchodilator for pulmonary administration

(76) Inventors: Leonard W. Kaplan, Brookline, MA (US); Karl P. Weinrich, Bridgewater, MA (US); Theodore L. Iorio, Milis, MA (US)

Correspondence Address: REED & ASSOCIATES, 800 MENLO AVENUE, SUITE 210, MENLO PARK, CA, 94025, US

(21) Appl. No.: 09/920,340

(22) Filed: Aug. 1, 2001

Related U.S. Application Data
(60) Provisional application No. 60/223,541, filed on Aug. 4, 2000.

(51) Int. Cl. A61K 9/00; A61K 31/57

(52) U.S. Cl. 424/43; 514/179

(57) ABSTRACT

A pharmaceutical formulation is provided for pulmonary drug administration of a bronchodilator, a corticosteroid and an optional pharmaceutically acceptable carrier. In addition, methods for using the formulation to treat bronchodilator/corticosteroid-responsive conditions, diseases or disorders are provided, as are drug delivery devices and dosage forms for housing and/or dispensing the formulations.

### CROSS REFERENCE TO RELATED APPLICATION

This application claims priority under 35 U.S.C. § 119(e)(1) to U.S. Provisional Application Serial No. 60/223,541, filed Aug. 4, 2000.

### TECHNICAL FIELD

This invention relates generally to pharmaceutical formulations, and more particularly relates to pharmaceutical formulations comprising a bronchodilator, corticosteroid and optional carrier. In addition, the invention relates to methods of using the described formulations and drug delivery devices containing the described formulations.

### BACKGROUND ART

Bronchodilators, particularly selective β₂ adrenergic agonists, e.g., albuterol, metaproterenol, salmeterol and terbutaline, are widely administered to individuals suffering from asthma. Most bronchodilators exert their anti-asthmatic effect by relaxing the smooth muscle tissue surrounding a constricted airway. Albuterol sulfate, for example, is a particularly effective bronchodilator and is used to relieve asthmatic symptoms such as shortness of breath and difficulty breathing. Because they are safe, bronchodilators can be, and often are, administered to young children.

Patients experiencing an asthmatic attack often require immediate relief to prevent further complications, e.g., hypoxia and even death. Therefore, it is critical that the bronchodilator exerts its pharmacological actions as quickly as possible. Inhalation therapy with a bronchodilator provides for a relatively fast onset of action by placing the drug in direct contact with the target tissues, i.e., pulmonary tissues. Consequently, bronchodilators are generally administered via a metered-dose inhaler for oral inhalation, although other vehicles, e.g., tablets and syrups, are also available. Thus, oral inhalation of bronchodilators offers the asthmatic patient a safe and effective means to relieve the symptoms of asthma.

Bronchodilator therapy, however, is not without drawbacks. For example, bronchodilators relieve only the immediate symptoms of asthma, e.g., shortness of breath and difficulty breathing. Thus, monotherapy with a bronchodilator will not decrease the number of asthmatic episodes a predisposed individual will experience.

In view of their many advantages, bronchodilators have been combined with other active agents in an attempt to reduce the number of asthmatic episodes. In particular, bronchodilators have been administered in combination with a corticosteroid. Examples of these combinations include: β₂ agonists in combination with glucocorticosteroids, U.S. Pat. No. 6,030,604 to Trofast; bronchodilators in combination with beclomethasone dipropionate monohydrate, U.S. Pat. No. 5,688,782 to Neale et al.; salmeterol in combination with fluticasone, Chapman et al. (1999) Can. Respir. J. 6(1):45-51; and salmeterol in combination with beclomethasone, Kelson et al. (1999) J. of Asthma 36(8):703-715. Because inflammation often triggers an attack of asthma, administration of an anti-inflammatory corticosteroid reduces the number of asthmatic attacks an individual may experience. Thus, the combination of a bronchodilator and corticosteroid not only provides immediate relief of asthmatic symptoms, but reduces the likelihood of a subsequent asthmatic attack as well.

Corticosteroid administration, however, presents the clinician with complications unique to this class of drugs. In particular, many corticosteroids are known to suppress the hypothalamic-pituitary-adrenal axis (HPA axis). Briefly stated, the HPA axis regulates the release of the body's own corticosteroids. Exogenous sources of corticosteroids induce a “negative feedback inhibition” in which the body slows production of endogenous corticosteroids. Once the exogenous source of corticosteroids ends, however, a delay occurs before the body's HPA axis returns to a normal balance. This delay results in a deficiency of corticosteroids in the body causing many side effects such as nausea, corticoid withdrawal symptoms and an increase in capillary fragility.

Mometasone furoate, however, is a corticosteroid that does not cause HPA axis suppression. Suppression of the HPA axis has not been reported in mice that have inhaled mometasone furoate. Chapman et al. (1998) Arzneimittelforschung 48(4):384-391. In addition, inhaled mometasone furoate did not cause HPA axis suppression in humans at dosages of up to 800 mcg daily. Bernstein et al. (1999) Respir. Med. 93(9):603-612.

Mometasone furoate, i.e., (11β, 16α)-9,21-dichloro-17-[(2-furanylcarbonyl)oxy]-11-hydroxy-16-methylpregna-1,4-diene-3,20-dione, has been described in U.S. Pat. No. 4,472,393 as an anti-inflammatory agent.

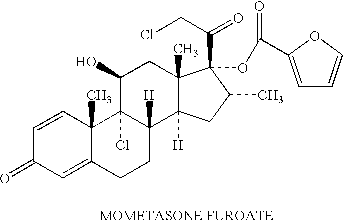

In addition, U.S. Pat. No. 6,057,307 describes mometasone furoate for treating corticosteroid-responsive diseases of the lungs and airway passages. Lumry discusses the clinical and pharmacological advantages of mometasone furoate in treating seasonal allergic rhinitis via nasal inhalation. Lumry (1999) J. Allergy Clin. Immunol. 104(4 Pt 1):S150-158. Nayak et al. describes inhaled administration of mometasone furoate once daily as effective in treating patients with mild to moderate persistent asthma. Nayak et al. (2000) Ann. Allergy Asthma Immunol 84(4):417-424. Each of these references, however, describes administering mometasone furoate as a single agent.

There is, accordingly, a need in the art to provide a composition for administering a bronchodilator/corticosteroid combination that will not cause suppression of the HPA axis. The present invention addresses both this and other needs in the art by providing a pharmaceutical formulation with minimal or no effect on the HPA axis. Specifically, the pharmaceutical formulation comprises a bronchodilator, a corticosteroid with minimal or no effect on the HPA axis, e.g., mometasone furoate, and optionally, a pharmaceutically acceptable carrier.

### DISCLOSURE OF THE INVENTION

Accordingly, it is a primary object of the invention to provide a pharmaceutical formulation for pulmonary drug administration comprising a composition of: a therapeutically effective amount of a bronchodilator; a therapeutically effective amount of a corticosteroid selected from the group consisting of mometasone and pharmacologically acceptable salts, esters and derivatives thereof; and optionally, a pharmaceutically acceptable carrier suitable for pulmonary drug administration.

It is another object of the invention to provide such a formulation wherein the bronchodilator has agonist activity for β₂ adrenergic receptors.

It is still another object of the invention to provide such a formulation in which the bronchodilator is pirbuterol acetate, pirbuterol dihydrochloride, levalbuterol sulfate or levalbuterol hydrochloride.

Another object of the invention is to provide a method for treating a patient suffering from a condition, disease or disorder that is responsive to treatment with a bronchodilator/corticosteroid combination by administering to the patient, via inhalation, a pharmaceutical formulation as provided herein.

Still another object of the invention is to provide a drug delivery device for pulmonary delivery of a pharmaceutical formulation as provided herein.

It is still yet another object of the invention to provide a dosage form containing a pharmaceutical formulation as provided herein.

Additional objects, advantages and novel features of the invention will be set forth in part in the description that follows, and in part will become apparent to those skilled in the art upon examination of the following, or may be learned by practice of the invention.

In one embodiment, a pharmaceutical formulation for pulmonary drug administration is provided, comprising a composition of: a therapeutically effective amount of a bronchodilator; and a therapeutically effective amount of a corticosteroid selected from the group consisting of mometasone and pharmacologically acceptable salts, esters and derivatives thereof. Optionally, the pharmaceutical formulation contains a pharmaceutically acceptable carrier suitable for pulmonary drug administration. Any bronchodilator, including pharmacologically acceptable salts and esters thereof, as well as combinations of bronchodilators suitable to treat asthma or asthmatic conditions, may be included in the formulation. It is preferred, however, that the bronchodilator present in the formulation has agonist activity for β₂ adrenergic receptors. Bronchodilators in this class include, for example, pirbuterol acetate, pirbuterol dihydrochloride, levalbuterol sulfate, and levalbuterol hydrochloride.

Although any salt, ester or derivative of mometasone may serve as the corticosteroid, it is particularly preferred that an ester form, e.g., acetate form, thiophene ester form or furoate form, of mometasone is present in the formulation. For the corticosteroid component, mometasone furoate is most preferred in the formulations described herein.

In another embodiment, a method for treating a patient suffering from a condition, disease or disorder that is responsive to treatment with a bronchodilator/corticosteroid combination is provided by administering to the patient, via inhalation, a pharmaceutical formulation as described herein. The formulations are particularly well suited to treat patients suffering from asthma, exercise-induced asthma, bronchitis, bronchospasm, rhinitis and emphysema. The formulations are effective in the treatment of patients suffering from both acute and chronic episodes of these maladies.

In yet another embodiment, a drug delivery device is provided comprising a pharmaceutical formulation as described herein and a means for housing and dispensing unit dosages of the formulation. The drug delivery device may be any device that is effective in delivering the formulation to the pulmonary system. Thus, for example, the drug delivery device may be a dry powder inhaler, a metered-dose inhaler (MDI), a nebulizer or a pump spray bottle. A dry powder inhaler is a particularly preferred device for delivering the formulations of the invention.

### BRIEF DESCRIPTION OF THE DRAWINGS

FIG. 1 is a cross-sectional side view of a preferred dry powder inhaler for administering the formulations of the invention.

FIG. 2 is a side view of the inhaler of FIG. 1, inverted so as to be positioned for delivering drug.

### MODES FOR CARRYING OUT THE INVENTION

I. Overview and Definitions

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a bronchodilator” includes a combination of two or more bronchodilators, reference to “a corticosteroid” includes combinations of two or more corticosteroids, reference to “a pharmaceutically acceptable carrier” includes combinations of two or more pharmaceutically acceptable carriers, and the like.

In describing and claiming the present invention, the following terminology will be used in accordance with the definitions set forth below.

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect.

By “pharmaceutically acceptable carrier” is meant a material or materials that are suitable for pulmonary drug administration and not biologically or otherwise undesirable, i.e., that may be administered to an individual along with an active agent without causing any undesirable biological effects or interacting in a deleterious manner with any of the other components of the pharmaceutical formulation in which it is contained.

Similarly, a “pharmacologically acceptable” salt, ester or other derivative of an active agent as provided herein is a salt, ester or other derivative that is not biologically or otherwise undesirable.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” asthma, as the term “treating” is used herein, encompasses both prevention of asthma in a predisposed individual and treatment of asthma in a clinically symptomatic individual.

The terms “condition,” “disease” and “disorder” are used interchangeably herein as referring to a physiological state that can be prevented or treated by administration of a pharmaceutical formulation as described herein.

The term “patient” as in treatment of “a patient” refers to a mammalian individual afflicted with or prone to a condition, disease or disorder as specified herein, and includes both humans and animals.

The term “pulmonary” as used herein refers to any part, tissue or organ that is directly or indirectly involved with gas exchange, i.e., O₂/CO₂ exchange, within a patient. “Pulmonary” contemplates both the upper and lower airway passages and includes, for example, the mouth, nose, pharynx, oropharynx, laryngopharynx, larynx, trachea, carina, bronchi, bronchioles and alveoli. Thus, the phrase “pulmonary drug administration” refers to administering the formulation described herein to any part, tissue or organ that is directly or indirectly involved with gas exchange within a patient.

“Optional” or “optionally” means that the subsequently described event or circumstance may or may not occur, and that the description includes instances where said event or circumstance occurs and instances where it does not. For example, reference to an “optional pharmaceutically acceptable carrier” in a formulation indicates that such a carrier may or may not be present, and the description includes formulations wherein a carrier is present and formulations wherein a carrier is not present.

II. The Pharmaceutical Formulations

The invention, as noted above, is in one embodiment a pharmaceutical formulation for pulmonary drug administration, comprising: a bronchodilator; a corticosteroid selected from the group consisting of mometasone and pharmacologically acceptable salts, esters and derivatives thereof, for example, as described in U.S. Pat. No. 4,472,393 to Shapiro; and, optionally, a pharmaceutically acceptable carrier suitable for pulmonary drug administration. Thus, the formulations described herein include at least two active agents, i.e., a bronchodilator and a corticosteroid selected from the group consisting of mometasone and pharmacologically acceptable salts, esters and derivatives thereof.

The formulation of the present invention may also contain various excipients, provided such excipients do not have a deleterious effect on the intended patient or have a deleterious chemical or physical effect on any component in the formulation. Thus, for example, excipients such as preservatives, surface active agents, buffering agents, suspending agents, and the like can be combined with the formulation. The type and amount of any excipient will depend on the type of formulation and the device used for administration, as will be appreciated by one of ordinary skill in the art. Specific examples of each of these excipients are well known by those skilled in the art of pharmaceutical formulation.

### A. ACTIVE AGENTS

Any of the active agents in the formulation may be administered in the form of a pharmacologically acceptable salt, ester, amide, prodrug or derivative or as a combination thereof. Salts, esters and derivatives of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol and the acid is added at a temperature of about 0° C. to about 100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, or the like. Preparation of esters involves functionalization of hydroxyl and/or carboxyl groups that may be present within the molecular structure of the drug. The esters are typically acyl-substituted derivatives of free alcohol groups, i.e., moieties which are derived from carboxylic acids of the formula RCOOH where R is alkyl, and preferably is lower, i.e., C₁ to C₆, alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

Stereoisomers of the active agents are also included as part of the formulations described herein. A stereoisomer is a compound having the same molecular weight, chemical composition, and constitution as another, but with the atoms arranged differently. That is, certain identical chemical moieties are at different orientations in space. This difference has the consequence of rotating the plane of polarized light. A pair of stereoisomers that are mirror images of each other are defined as enantiomers. Individual stereoisomers or enantiomers may have unique or beneficial properties that make that individual isomer particularly well suited for the present invention. Consequently, individual stereoisomers or enantiomers and mixtures thereof of the active agents are included as part of the invention. Thus, each active agent may be present in the formulation as a racemate, i.e., equal amounts of each enantiomer, an enantiomerically pure form, e.g., levalbuterol, or a mixture of nonequal amounts of each enantiomer, e.g., nonequal amounts of (S)-albuterol/(R)-albuterol.

The various hydrates of the active agents are also included in the formulations of the invention. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, exposing the active agent to dry heat.

The invention is not limited with respect to the bronchodilator. Bronchodilators from the pharmacological classes of β₂ adrenergic agonists, anticholinergics and xanthine derivatives may be incorporated into the formulations. It is preferred, however, that the bronchodilator has agonist activity for β₂ adrenergic receptors. Furthermore, the formulation is not limited to one bronchodilator as combinations of bronchodilators may also be present.

Typical bronchodilators of the β₂ adrenergic agonist class include, but are not limited to, albuterol, bitolterol, clenbuterol, fenoterol, formoterol, levalbuterol (i.e., homochiral (R)-albuterol), metaproterenol, pirbuterol, procaterol, reproterol, rimiterol, salmeterol and terbutaline. The bronchodilator may be present in the formulation as a salt, ester, amide, prodrug, or other derivative, or may be functionalized in various ways as will be appreciated by those skilled in the art.

Preferred β₂ adrenergic agonists are pirbuterol, levalbuterol, metaproterenol, derivatives thereof, pharmacologically acceptable salts and esters thereof, and combinations of any of the foregoing. More preferred, however, are pirbuterol acetate, pirbuterol dihydrochloride, levalbuterol sulfate and levalbuterol hydrochloride. When levalbuterol sulfate is incorporated into the formulations described herein, it is preferred that levalbuterol sulfate is formulated in a dry powder formulation. When levalbuterol hydrochloride is the bronchodilator of the formulation, it is preferred that the formulation is an aerosol or liquid.

Any corticosteroid selected from the group consisting of mometasone and its pharmacologically acceptable salts, esters and derivatives thereof may be included in the present formulation. Pharmacologically acceptable esters of mometasone are preferred, with those derived from C₂ to C₆ carboxylic acids being particularly preferred. More specifically, the esters may be derived from corresponding carboxylic acids having two to six carbon atoms that are branched, unbranched or cyclic, saturated or unsaturated, aromatic or nonaromatic, and heteroatom substituted or unsubstituted. It is particularly preferred, however, that the ester derivative of mometasone is mometasone furoate, mometasone thiophene ester, or mometasone monoacetate, with mometasone furoate most preferred. Furthermore, it is preferred that anhydrous mometasone furoate is present in the formulation when the formulation is a dry powder. Mometasone furoate monohydrate is preferred when the formulation is an aerosol or liquid, e.g., an aqueous suspension.

The formulations of the present invention may take any form suitable for delivering the active agents to a patient. For example, the formulations may be in the form of a dry powder, aerosol or liquid.

### B. DRY POWDER FORMULATIONS

The dry powder formulations as described herein include, at a minimum, both the bronchodilator and corticosteroid. Such dry powder formulations can be administered via pulmonary inhalation to a patient without the benefit of a carrier. Preferably, dry powders formulations that do not include a carrier are administered with the aid of, for example, a dry powder inhaler as described in section IV, infra.

Preferably, however, the dry powder formulations described herein include one or more pharmaceutically acceptable carriers. Although any carrier suitable for pulmonary drug administration may be used, pharmaceutical sugars are particularly preferred for use as carriers in the present invention. Preferred pharmaceutical sugars include those selected from the group consisting of fructose, galactose, glucose, lactitol, lactose, maltitol, maltose, mannitol, melezitose, myoinositol, palatinite, raffinose, stachyose, sucrose, trehalose, xylitol, hydrates thereof, and combinations of any of the foregoing. It is particularly preferred, however, that lactose, e.g., lactose U.S.P., serves as the carrier in the present invention when the formulation is a dry powder.

Once selected, each active agent or the active agents in combination are blended to form a substantially homogeneous powder mixture. Techniques involved with the preparation of such powders are well known in the art. Briefly stated, however, the preparation generally includes the steps of reducing the particle size of each active agent (again, alone or in combination), and blending. Of course, reducing the particle size of each active agent is not required when a commercially available product having a suitable particle size is used. Techniques for reducing the particle size include, for example, using mills such as an air-jet mill or a ball mill. The active agents should have a particle size diameter of between about 0.1 μm to about 65 μm for pulmonary administration. It is preferred that the active agent particles are about 1 μm to about 10 μm, more preferably about 2 μm to about 5 μm in diameter.

Similarly, the particle size of the remaining components, e.g., carrier, excipient, etc., must be controlled as well. The same techniques described above for reducing the particle size of active agents may be used to reduce the particle size of the remaining components. Again, such techniques are not required when the component is available commercially in the desired particle size range. Preferably, the remaining components, particularly the carrier, have a particle size from about 30 μm to about 100 μm in diameter, with sizes from about 30 μm to about 70 μm most preferred.

For any given particle size range, it is preferred that at least about 60%, more preferably at least about 70%, still more preferably at least about 85%, of the stated particles have a size within the stated or given range. It is most preferred, however that at least about 90% of the particles have the size in the stated or given range. For example, when a component is stated to have a particle size less than 10 μm, it is most preferred that at least 90% of the particles of that component have a particle size of less than 10 μm.

As previously stated, some components of the formulation may be commercially available in the desired particle size range. For example, a preferred lactose product for use in some embodiments of the present invention is the PHARMATOSE™ 325 brand of lactose monohydrate available from DMV International, Veghel, The Netherlands. According to the manufacturer, 100% of the lactose particles have a particle size of less than 100 μm, and only 5 to 10% of the particles have a particle size of less than 32 μm. Furthermore, a minimum of 70% of the lactose particles are stated to have a particle size of less than 63 μm. Advantageously, particle size manipulation steps are avoided when components are commercially available in the desired particle size range.

Preferably, the particle size reduction of the active agents and the particle size reduction of the remaining components are carried out separately. In this way, it is possible to provide a formulation in which the particle size of the active agents is smaller than the particle size of, for example, the carrier. The advantage of such a formulation is that the active agents penetrate deeply into the pulmonary tract while the carrier (having a relatively larger particle size) is retained in the upper airways.

Conventional blending techniques known to those skilled in the art may be used for combining active agents or for combining the active agents with the carrier and/or remaining components. Such blending techniques include passing the combined powders through a sifter or blending, for example, the active agents and carrier in a powder blender such as a “double cone” blender or a “V-blender.” No matter which technique is employed, however, it is necessary that the resulting powder is a substantially homogeneous mixture. Typically, the active agents will make up from about 0.01% to about 99% of the total formulation, preferably from about 0.05% to 50% of the total formulation by weight.

After blending, the powder formulation may, if desired, be portioned and/or otherwise processed into unit dose quantities, e.g., portioned into unit dose quantities and individually placed within a dosage form or drug delivery system. Alternatively, the powder formulation may be loaded into a dosage form or drug delivery device and not “metered out” into unit doses until used. Although any dosage form that contains a unit dose of the formulation is acceptable, capsules are preferred. The capsule material may be either hard or soft, and, as will be appreciated by those skilled in the art, typically comprises a water-soluble compound such as gelatin, starch or a cellulosic material. Preferably, the capsules are composed of a cellulosic material, e.g., hydroxypropyl methylcellulose (HPMC). The capsules may be sealed, such as with gelatin bands or the like. See, for example, Remington: The Science and Practice of Pharmacy, Twentieth Edition (Easton, Pa.: Mack Publishing Co., 2000), which describes materials and methods for preparing encapsulated pharmaceuticals. Thus, each capsule or dosage form will typically contain a therapeutically effective dose of each active agent

Alternatively, the dosage forms may contain less than a therapeutically effective dose in which case administration of two or more dosage forms would be required to achieve the therapeutically effective dose.

### C. AEROSOL FORMULATIONS

The formulations of the present invention may also take the form of an aerosol composition for inhalation. Aerosol formulations are known to those skilled in the art and are described in Remington: The Science and Practice of Pharmacy, supra. Briefly, the aerosol formulation of the invention is either a solution aerosol in which the active agents are soluble in the carrier (e.g., propellant) and optional solvent or a dispersion aerosol in which the active agents are suspended or dispersed throughout the carrier and optional solvent. It is preferred that the aerosol formulations of the invention are in the form of a dispersion aerosol.

The carrier in the aerosol formulations of the invention is generally a propellant, usually a compressed gas, e.g., air, nitrogen, nitrous oxide, and CO₂, a mixture of compressed gases, a liquefied gas or a mixture of liquefied gases. A mixture of propellants, when present in the formulations, may be comprised of two, three, four or propellants. Preferred mixtures of propellants, however, comprise only two propellants. Any propellant used in the art of preparing aerosol formulations may be used.

Typically, the propellant is a chlorofluorocarbon, a hydrochlorofluorocarbon, a hydrogen-containing fluorocarbon, a perfluorocarbon, a hydrocarbon or a mixture thereof. Preferably, the propellant is a hydrochlorofluorocarbon, a hydrogen-containing fluorocarbon, a perfluorocarbon or a mixture thereof.

Preferred chlorofluorocarbons include dichlorotetrafluoroethane (e.g., CClF₂CClF₂ and CCl₂FCF₃), trichloromonofluoromethane, dichlorodifluoromethane, chloropentafluoroethane, and mixtures thereof. Preferred hydrochlorofluorocarbons include monochlorodifluoromethane, monochlorodifluoroethane (e.g., 1-chloro-1,1-difluoroethane), and mixtures thereof. Preferred hydrogen-containing fluorocarbons include C₁₋₄hydrogen-containing fluorocarbons such as CHF₂CHF₂, 1,1,1,2-tetrafluoroethane (HFA-134a), difluoroethane (e.g., 1,1-difluoroethane), 1,1,1,2,3,3,3-heptafluoropropane (HFA-227), and mixtures thereof. Preferred perfluorocarbons include CF₃CF₃, CF₃CF₂CF₃, octafluorocyclobutane, and mixtures thereof. Preferred hydrocarbons include propane, isobutane, n-butane, dimethyl ether, and mixtures thereof. Most preferably, the propellant is selected from the group consisting of difluoroethane, CHF₂CHF₂, 1,1,1 ,2-tetrafluoroethane, 1,1,1,2,3,3,3-heptafluoropropane, CF₃CF₃, CF₃CF₂CF₃, octafluorocyclobutane, and mixtures of any of the foregoing.

As will be appreciated by one skilled in the art, the aerosol formulations of the invention may include one or more excipients. For example, the aerosol formulations may contain: a solvent (e.g., water, ethanol and mixtures thereof) for increasing the solubility of the active agent; an antioxidant (e.g., ascorbic acid) for inhibiting oxidative degradation of the active agents; a dispersing agent (e.g., sorbitan trioleate, oleyl alcohol, oleic acid, lecithin, e.g., soya lecithin, corn oil, or combinations thereof) for preventing agglomeration of particles; and/or a lubricant (e.g., isopropyl myristate) for providing slippage between particles and lubricating the components, e.g., the valve and spring, of the inhaler.

As described with respect to dry powder formulations in Section B, the particle size released from aerosol formulations must be appropriate for pulmonary administration. Solution aerosols inherently produce small particles upon actuation of the inhaler given that the active agent is expelled along with the carrier, i.e., propellant, solution as it evaporates. Consequently, solution aerosols produce sufficiently small particles, e.g., within a range of about 0.1 μm to about 65 μm, of active agents upon administration. In contrast, dispersion aerosols contain undissolved active agents in which particle size remains constant, i.e., the size of the particles in the dispersion aerosol remains unchanged as the active agent is delivered to the patient. Thus, the active agents must have an appropriate particle size before being formulated into a dispersion aerosol. Consequently, methods of reducing the particle size of the active agents for the dry powder formulations described above are equally applicable for preparing active agents with an appropriate particle size in a dispersion aerosol. Furthermore, the same ranges of particle sizes preferred for the dry powder formulations are equally applicable for dispersion aerosols.

The aerosol formulation may be prepared by employing a cold filling process. Initially, the components of the aerosol formulation and an aerosol container are cooled, e.g., to about −40° C., such that the carrier, i.e., propellant, is a liquid. All components except for the carrier are placed into the aerosol container. Thereafter, the carrier is added, the components mixed, and a valve assembly inserted into place. The valve assembly is then crimped such that the container is airtight. Thereafter, the container and formulation contained therein are allowed to return to ambient temperature.

As an alternative to the cold filling process, the aerosol formulation may be prepared by transfer of a carrier from a bulk container. In such a process, the components except for the carrier are initially placed into an empty aerosol container. A valve assembly is then inserted and crimped into place. The carrier, under pressure and in liquid form, is metered through the valve assembly from a bulk container or tank of carrier. The container housing the formulation is checked to ensure that the pressurized contents do not leak.

For both of these methods of preparing the aerosol formulations, the active agents generally represent from about 0.1 wt. % to about 40 wt. % of the total formulation. It is preferred, however, that the active agents represent about 2 wt. % to about 20 wt. % of the total formulation, with 5 wt. % to about 15 wt. % being most preferred.

### D. LIQUID FORMULATIONS

The formulations of the present invention may also take the form of a liquid composition for inhalation. Liquid formulations are well known in the art. See, for example, Remington: The Science and Practice of Pharmacy, supra. It is preferred that the liquid is an aqueous suspension, although aqueous solutions may be used as well. The liquid formulations include one or more carriers in addition to the active agents. Generally, the carrier is a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Combining the components followed by conventional mixing results in a liquid formulation suitable for inhalation. Typically, the active agents will make up from about 0.01% to about 40% of the total formulation.

III. Utility and Administration

The invention provides a method for treating a patient suffering from or prone to a condition, disease or disorder that is responsive to treatment with a bronchodilator/corticosteroid combination, comprising administering to the patient, via inhalation, a pharmaceutical formulation for pulmonary drug administration, wherein the formulation comprises: a bronchodilator; a corticosteroid selected from the group consisting of mometasone and pharmacologically acceptable salts, esters and derivatives thereof; and optionally, a pharmaceutically acceptable carrier suitable for pulmonary drug administration. For example, the method is particularly advantageous for treating patients suffering from asthma (including exercised-induced asthma), bronchitis, bronchospasm, rhinitis and emphysema.

The formulations as described herein have many advantages over conventional inhalation formulations. Because the pharmaceutical formulation combines two active agents, patients receive the benefits of two active agents with only one formulation. As a further consequence of combining two active agents, the formulation of the present invention increases patient compliance as the likelihood of missed doses of a second active agent is eliminated. In addition, mometasone (or its salt, ester or derivative thereof) does not, as previously indicated, cause suppression of the HPA axis. Furthermore, particular ingredients may have additional advantages when present in the formulation. For example, pirbuterol and levalbuterol do not cause side effects such as excitability and heart tremors that may be caused by other bronchodilators. Finally, the patient may only need to administer the formulation on an “as-needed” basis because the bronchodilator acts to relieve symptoms immediately while the mometasone component acts to treat the underlying inflammation causing the asthmatic symptom(s). In this way, the formulation addresses both the symptoms and causes of an asthmatic attack, thereby obviating the need to administer repeated doses of a drug that only treats the symptoms of an asthmatic attack. Consequently, daily administration may not be necessary.

Additional advantages result when the formulations are administered via the preferred dry powder inhalers described in section IV, infra. Such dry powder inhalers assure that patients, particularly those patients that have traditionally had trouble using inhalers such as children or the elderly, obtain the complete dose. Even medical personnel who are responsible for monitoring and instructing patients in optimal inhaler use lack the rudimentary skills associated with MDIs. See Hanania et al. (1994) Chest 105(l):111-116. Administration of the complete dose is ensured with these preferred dry powder inhalers since little effort in inhalation is required in order to deliver all of the dose to the lungs. This is in contrast to, for example, metered-dose inhalers with which patients must coordinate the actuation of the inhaler with a deep and prolonged inhalation to ensure that the entire dose is received. As a result of the foregoing advantages, the dry powder inhalers described herein may be efficient in delivering the present formulations in reduced dosages, i.e., 5% to 15% less than the dose used in conventional devices.

The actual amount of each active agent in the formulation will, of course, depend upon the age, weight, and general condition of the subject, the severity of the condition being treated, and the judgment of the prescribing physician. Therapeutically effective amounts are known to those skilled in the art and/or are described in the pertinent reference texts and literature. An effective amount of the formulation may be administered with a single administration, e.g., serially administration of the contents of a single capsule containing a therapeutically effective amount of the formulation via a dry powder inhaler or a single actuation of an aerosol inhaler designed to deliver a therapeutically effective amount of the formulation. Alternatively, a patient can obtain an effective amount of the formulation by, for example, administering multiple doses, e.g., serially administering the contents of multiple capsules containing the formulation via a dry powder inhaler.

Furthermore, the actual amount of each active agent will also depend on particular “synergies” between the two active agents. That is, certain combinations and/or ratios of the bronchodilator/corticosteroid combinations described herein provide enhanced treatment of a particular condition.

For the bronchodilator, for example, the formulation will be prepared such that each dose (or administration) of the formulation will deliver the bronchodilator in a therapeutically effective amount, typically in the range of about 1 μg to about 1500 μg. When the bronchodilator is pirbuterol acetate or pirbuterol dihydrochloride, for example, a suitable dosage is in the range of about 2.5 μg to about 350 μg, preferably from 50 μg to about 100 μg. When the bronchodilator is levalbuterol sulfate, a suitable dosage is in the range of about 5 μg to about 150 μg, preferably from about 50 μg to about 100 μg. When the bronchodilator is levalbuterol hydrochloride, a suitable dose is in the range of about 50 μg to about 1300 μg, preferably from about 600 μg to about 1000 μg. When the corticosteroid is anhydrous mometasone furoate or mometasone furoate monohydrate, a suitable dosage is in the range of about 1 μg to about 1500 μg, preferably from about 25 μg to about 100 μg.

The formulations may be administered in a variety of dosing regimens including: as-needed administration; one, two, three or four administrations once daily; one, two, three or four administrations twice daily; one, two, three or four administrations three times daily; and one, two, three or four administrations four times daily. Generally, however, the total daily dose of the bronchodilator should not exceed about 5000 μg and the total daily dose of the corticosteroid (i.e., mometasone) should not exceed about 8000 μg.

The formulations of the invention may be administered via oral or nasal inhalation. For oral administration, the patient inhales the formulation through the mouth. The inhaled formulation progressively comes into contact with the air passages of the mouth and throat area, the upper respiratory tract, e.g., trachea, and finally the lower respiratory tract, e.g., bronchioles. The corticosteroid, i.e., mometasone moiety, acts to decrease inflamed and congested air passages, thereby facilitating gas exchange by increasing the diameter of the air passages. The bronchodilator, e.g., β₂ agonist, acts to relax bronchial smooth muscle, which also facilitates gas exchange by opening up closed or constricted passages. Nasal inhalation is similar to oral inhalation except that the patient inhales the formulation through the nares, preferably one at a time. For example, the formulation may be administered via a pump spray in which the patient administers a spray in the left nare followed by administration in the right nare. Nasal administration provides an added benefit of relieving nasal congestion (if present) in that the corticosteroid is placed in contact with nasal tissue.

IV. Inhalers

The invention also provides a dry powder inhaler containing a formulation as described herein. Dry powder inhalers are well known to those skilled in the art. Preferably, the dry powder inhaler includes at least one capsule (preferably a hydroxypropyl methylcellulose capsule) containing a unit dose of the formulation. The patient self-administers the dose by inhaling (via oral or nasal inhalation) the dry powder formulation from the inhaler. In this manner, delivery of the dry powder formulation to the pulmonary system is effected.

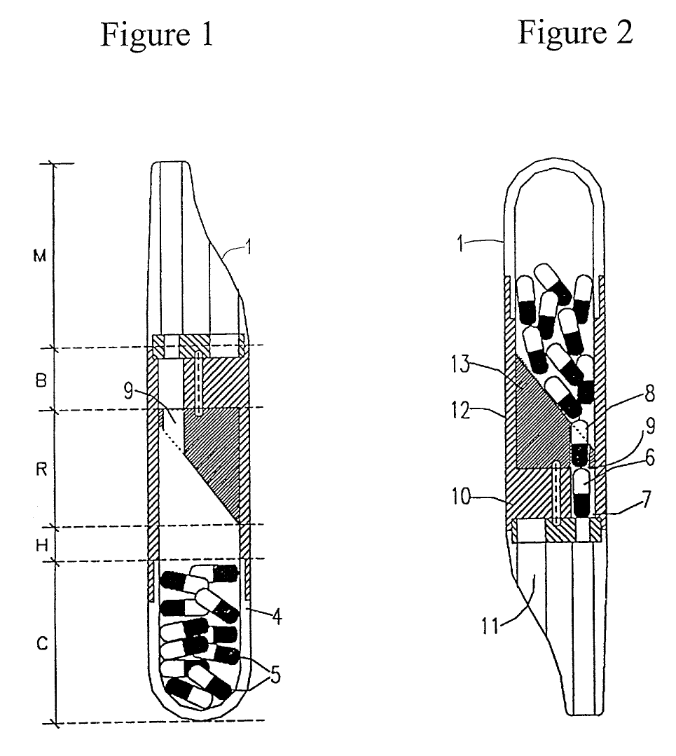

One example of a particularly preferred dry powder inhaler is described in U.S. Pat. Nos. 5,673,686 to Villax et al. and 5,881,721 to Bunce et al. Specifically, as shown in FIG. 1, a dry powder inhaler 1 comprises a mouthpiece M, a barrel area B, a ramp area R, free headspace H and a capsule container area C. The capsule container 4 is filled to the brim with capsules 5. FIG. 2 shows the same inhaler 1 which has been inverted. The capsules now fill the free headspace and the ramp area and become vertically oriented as they near the passage 9. One capsule 8 is already inserted into the passage 9 and its movement is blocked by the capsule 6 which has preceded it and been dispensed into the capsule chamber 7. The capsule chamber 7 is contained inside a rotating barrel 10.

The operation of the inhaler requires that once a capsule has been loaded into the capsule chamber 7, the rotating barrel 10 is turned. This movement transports the capsule 6 past two small blades (not shown), which slits both ends and carries the capsule to the inhalation position. Once inhalation has taken place, a further turn of the barrel 10 delivers the capsule to the ejection position 11. Continuing to turn the rotating barrel 10 brings the capsule chamber 7 in alignment again with the passage 9 where the next capsule 8 is in place for dispensing.

The rotating barrel 10 is connected to the cylindrical tube 12 and is unconnected to the ramp 13. In operation, the turning motion of the rotating barrel 10 and cylindrical tube 12 is in opposite direction to that of the ramp 13. These opposite turning motions further assist the righting of the capsules between the ramp 13 and the cylindrical tube 12 and dispensing of the capsule into the passage 9.

Thus, the dry powder inhaler comprises a tube, a ramp, and a dispensing passage. The tube receives a capsule or similar dosage unit that must be properly oriented. The ramp has a surface that extends substantially across the tube from one wall to an opposite wall. An elongate dispensing passage has a diameter less than that of the tube and is sized to receive the capsule to be dispensed, but only when the axis of the capsule is generally parallel to the axis of the passage. The elongate dispensing passage extends from an inlet end formed by an aperture in the ramp's surface to a dispensing outlet, the passage being adjacent to one wall of the tube such that the axis of the passage is parallel to, but radially offset from, an axis of the tube. The arrangement is such that when the apparatus is positioned with the passage below the tube and the axis of the passage is substantially vertical, a capsule located in the tube will be guided by the ramp surface towards the inlet end of the passage.

Once the capsule has been properly aligned and pierced, the patient inserts the end of the mouthpiece of the inhaler into his or her mouth and inhales. Air enters through the device via any path but generally though specialized air inlets (not shown) on the device. As air enters the inhaler, at least a portion is drawn through an upstream slit. As it travels through the upstream slit into the pierced capsule, the air fluidizes or entrains the powder in the pierced capsule creating what has been referred to as a “dancing cloud.” As suction continues from the patient, the powder-containing air exits through a downstream slit in the pierced capsule and enters the bore of the mouthpiece for passage into the patient's pulmonary system.

Additional dry powder inhalation devices suitable for administering the present invention include, for example, TURBUHALER® (Astra Pharmaceutical Products, Inc., Westborough, Mass.), ROTAHALER® and DISKHALER® devices (both available from Allen & Hanburys, Ltd., London, England). Aerosol formulations of the present invention may be administered via pressurized metered-dose inhalers. Liquid formulations of the invention may be administered via a pump spray bottle or nebulizer.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXPERIMENTAL

The practice of the present invention will employ, unless otherwise indicated, conventional techniques of pharmaceutical formulation and the like, which are within the skill of the art. Such techniques are explained fully in the literature.

In the following examples, efforts have been made to ensure accuracy with respect to numbers used (e.g., amounts, temperature, etc.) but some experimental error and deviation should be accounted for. Unless indicated otherwise, temperature is in degrees C and pressure is at or near atmospheric. All reagents were obtained commercially unless otherwise indicated.

### EXAMPLE 1

Pirbuterol acetate (10.0 mg), 10.0 mg of mometasone furoate (anhydrous) and 2000 mg of lactose are blended using conventional blending techniques to form a dry pharmaceutical formulation. Particle size reduction is not required as each of the components is obtained having a suitable particle size. The dry pharmaceutical formulation is then divided, in equal portions, into 100 capsules (capsule size 4). The amount of each component of the formulation per capsule is presented in Table 1.

TABLE 1

| Component | amount per capsule | |
|---|---|---|
| Pirbuterol acetate | 100 | μg |
| Mometasone furoate (anhydrous) | 100 | μg |
| Lactose | 20.00 | mg |

### EXAMPLE 2

Pirbuterol acetate (25.0 mg), 20.0 mg of mometasone furoate (anhydrous) and 2500 mg of lactose are blended using conventional blending techniques to form a dry pharmaceutical formulation. The dry pharmaceutical formulation is then divided, in equal portions, into 100 capsules (capsule size 4). The amount of each component of the formulation per capsule is presented in Table 1.

TABLE 2

| Component | amount per capsule | |
|---|---|---|
| Pirbuterol acetate | 250 | μg |
| Mometasone furoate (anhydrous) | 200 | μg |
| Lactose | 25.00 | mg |

### EXAMPLE 3

Levalbuterol sulfate (5.00 mg), 10.0 mg of mometasone furoate (anhydrous) and 2000 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 3.

TABLE 3

| Component | amount per capsule | |
|---|---|---|
| Levalbuterol sulfate | 50 | μg |
| Mometasone furoate (anhydrous) | 100 | μg |
| Lactose | 20.00 | mg |

### EXAMPLE 4

Levalbuterol sulfate (9.00 mg), 10.0 mg of mometasone furoate (anhydrous) and 2500 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 4.

TABLE 4

| Component | amount per capsule | |
|---|---|---|
| Levalbuterol sulfate | 90 | μg |
| Mometasone furoate (anhydrous) | 100 | μg |
| Lactose | 25.00 | mg |

### EXAMPLE 5

Levalbuterol sulfate (15.00 mg), 20.0 mg of mometasone furoate (anhydrous) and 3000 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 5.

TABLE 5

| Component | amount per capsule | |
|---|---|---|
| Levalbuterol sulfate | 150 | μg |
| Mometasone furoate (anhydrous) | 200 | μg |
| Lactose | 30.00 | mg |

### EXAMPLE 6

Levalbuterol hydrochloride (50.00 mg), 5.0 mg of mometasone furoate (anhydrous) and 2000 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 6.

TABLE 6

| Component | amount per capsule | |
|---|---|---|
| Levalbuterol hydrochloride | 500 | μg |
| Mometasone furoate (anhydrous) | 50 | μg |
| Lactose | 20.00 | mg |

### EXAMPLE 7

Levalbuterol hydrochloride (75.00 mg), 10.0 mg of mometasone furoate (anhydrous) and 2500 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 7.

TABLE 7

| Component | amount per capsule | |
|---|---|---|
| Levalbuterol hydrochloride | 750 | μg |
| Mometasone furoate (anhydrous) | 100 | μg |
| Lactose | 25.00 | mg |

### EXAMPLE 8

Levalbuterol hydrochloride (100.00 mg), 20.0 mg of mometasone furoate (anhydrous) and 3000 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 8.

TABLE 8

| Component | amount per capsule | |
|---|---|---|
| Levalbuterol hydrochloride | 1000 | μg |
| Mometasone furoate (anhydrous) | 200 | μg |
| Lactose | 30.00 | mg |

### EXAMPLE 9

Pirbuterol dihydrochloride (7.5 mg), 30.0 mg of mometasone furoate (anhydrous) and 2200 mg of lactose are blended using conventional blending techniques to form a dry pharmaceutical formulation. The dry pharmaceutical formulation is then divided, in equal portions, into 100 capsules (capsule size 4). The amount of each component of the formulation per capsule is presented in Table 9.

TABLE 9

| Component | amount per capsule | |
|---|---|---|
| Pirbuterol dihydrochloride | 75 | μg |
| Mometasone furoate (anhydrous) | 300 | μg |
| Lactose | 22.00 | mg |

### EXAMPLE 10

Pirbuterol dihydrochloride (25.0 mg), 20.0 mg of mometasone furoate (anhydrous) and 2000 mg of lactose are blended using conventional blending techniques to form a dry pharmaceutical formulation. The dry pharmaceutical formulation is then divided, in equal portions, into 100 capsules (capsule size 4). The amount of each component of the formulation per capsule is presented in Table 10.

TABLE 10

| Component | amount per capsule | |
|---|---|---|
| Pirbuterol dihydrochloride | 250 | μg |
| Mometasone furoate (anhydrous) | 200 | μg |
| Lactose | 20.00 | mg |

### EXAMPLE 11

A liquid suspension for inhalation is prepared. The percent amount of each component of the formulation is presented in Table 11.

TABLE 11

| | amount % |
|---|---|
| Component | (wt./wt.) |
| Pirbuterol acetate | 0.20% |
| Mometasone furoate monohydrate | 0.05%* |
| Glycerin | 2.1% |
| Microcrystalline cellulose and | 2.0% |
| carboxymethylcellulose sodium mixture, e.g., Avicel ® | |
| RC-591 (available from FMC, Corp. Philadelphia, PA) | |
| Sodium Citrate | 0.28% |
| Phenylethyl alcohol | 0.25% |
| Citric acid | 0.20% |
| Benzalkonium chloride | 0.20% |
| Oleate ester of sorbitol and its anhydride | 0.01% |
| copolymerized with ethylene oxide, e.g., polysorbate | |
| 80 (available from ICI Americas, Bridgewater, NJ) | |
| Water | q.s. |

*Equivalent to mometasone furoate calculated on the anhydrous basis.

Microcrystalline cellulose and the carboxymethylcellulose sodium mixture are dispersed in water followed by the addition of glycerin to form a dispersion. A solution of citric acid and sodium citrate in water is prepared and then added to dispersion. Separately, oleate ester of sorbitol and its anhydride copolymerized with ethylene oxide is dissolved in water and stirred. Both pirbuterol acetate and mometasone furoate monohydrate are added to the sorbitol solution and mixed to form a slurry. The slurry is then added to the dispersion with simultaneous stirring to form a suspension. Both the benzalkonium chloride and phenylethyl alcohol are dissolved in water and then added to the suspension with simultaneous stirring. Water is added to bring the suspension to 100%.

The liquid suspension is administered to a patient via a conventional pump spray bottle adapted for nasal inhalation. Following administration, the patient notes a decrease of allergy-induced bronchospasms.

### EXAMPLE 12

Example 11 is followed except that levalbuterol sulfate (0.09 wt./wt. %) is substituted for pirbuterol acetate.

### EXAMPLE 13

Example 11 is followed except that levalbuterol hydrochloride (0.750 wt./wt. %) is substituted for pirbuterol acetate.

### EXAMPLE 14

Example 11 is followed except that pirbuterol dihydrochloride (0.200 wt./wt. %) is substituted for pirbuterol acetate.

### EXAMPLE 15

A metered-dose inhaler is prepared. The percent amount of each component in the formulation is presented in Table 12.

TABLE 12

| Component | amount % wt./wt. |
|---|---|
| Pirbuterol acetate | 5-15% |
| Mometasone furoate (anhydrous) | 5-15% |
| Fluorinated hydrocarbon | 70-90% |
| (propellant/carrier) | |

The active components are placed into an empty aerosol container. Thereafter, a valve assembly is inserted into the aerosol container and crimped into place so as to provide an airtight seal. The propellant/carrier is then metered through the valve assembly from a tank of bulk propellant/carrier stored under pressure. The aerosol container is then placed in an adaptor suited for actuating aerosol containers and delivering metered amounts of the active agents to a patient. An asthmatic patient is relieved of her asthmatic symptoms upon pulmonary administration of the formulation via the metered-dose inhaler.

### EXAMPLE 16

The capsules made in any one of Examples 1 through 10 are placed in the dry powder inhaler as described in U.S. Pat. Nos. 5,673,686 to Villax et al. and 5,881,721 to Bunce et al. and as illustrated in FIGS. 1 and 2. Once the capsule has been properly aligned and pierced in the inhaler, a patient having an asthmatic attack inserts the mouthpiece of the inhaler into his mouth and inhales normally through the mouth. The inhalation causes the formulation to exit the pierced capsule and travel into the patient's pulmonary system. Relief of the asthmatic attack immediately results.

What is claimed is:

1. A pharmaceutical formulation for pulmonary drug administration, comprising:
a therapeutically effective amount of a bronchodilator;
a therapeutically effective amount of a corticosteroid selected from the group consisting of mometasone and pharmacologically acceptable salts, esters and derivatives thereof; and
optionally, a pharmaceutically acceptable carrier suitable for pulmonary drug administration.

2. The formulation of claim 1, wherein the bronchodilator has agonist activity for β₂ adrenergic receptors.

3. The formulation of claim 2, wherein the bronchodilator is selected from the group consisting of albuterol, bitolterol, clenbuterol, fenoterol, formoterol, levalbuterol, metaproterenol, pirbuterol, procaterol, reproterol, rimiterol, salmeterol, terbutaline, derivatives thereof, pharmacologically acceptable salts and esters thereof, and combinations of any of the foregoing.

4. The formulation of claim 3, wherein the bronchodilator is selected from the group consisting of pirbuterol, levalbuterol, metaproterenol, derivatives thereof, pharmacologically acceptable salts and esters thereof, and combinations of any of the foregoing.

5. The formulation of claim 4, wherein the bronchodilator is pirbuterol acetate.

6. The formulation of claim 4, wherein the bronchodilator is pirbuterol dihydrochloride.

7. The formulation of claim 4, wherein the bronchodilator is levalbuterol sulfate.

8. The formulation of claim 4, wherein the bronchodilator is levalbuterol hydrochloride.

9. The formulation of claim 1, in the form of a dry powder.

10. The formulation of claim 9, wherein the bronchodilator is pirbuterol or a pharmacologically acceptable salt thereof.

11. The formulation of claim 10, wherein the bronchodilator is pirbuterol dihydrochloride or pirbuterol acetate.

12. The formulation of claim 9, wherein the bronchodilator is levalbuterol or a pharmacologically acceptable salt thereof.

13. The formulation of claim 12, wherein the bronchodilator is levalbuterol sulfate.

14. The formulation of claim 9, wherein the corticosteroid is anhydrous mometasone furoate.

15. The formulation of claim 9, wherein the carrier is present.

16. The formulation of claim 15, wherein the carrier is selected from the group consisting of fructose, galactose, glucose, lactitol, lactose, maltitol, maltose, mannitol, melezitose, myoinositol, palatinite, raffinose, stachyose, sucrose, trehalose, xylitol, hydrates thereof, and combinations of any of the foregoing.

17. The formulation of claim 16, wherein the carrier is lactose.

18. The formulation of claim 9, wherein the carrier is not present.

19. The formulation of claim 1, wherein the carrier is present.

20. The formulation of claim 19, in the form of an aerosol composition.

21. The formulation of claim 20, wherein the bronchodilator is pirbuterol or a pharmacologically acceptable salt thereof.

22. The formulation of claim 21, wherein the bronchodilator is pirbuterol dihydrochloride or pirbuterol acetate.

23. The formulation of claim 20, wherein the bronchodilator is levalbuterol or a pharmacologically acceptable salt thereof.

24. The formulation of claim 23, wherein the bronchodilator is levalbuterol hydrochloride.

25. The formulation of claim 20, wherein the corticosteroid is mometasone furoate monohydrate.

26. The formulation of claim 20, wherein the carrier is a propellant.

27. The formulation of claim 26, wherein the propellant is selected from the group consisting of a chlorofluorocarbon, a hydrochlorofluorocarbon, a hydrogen-containing fluorocarbon, a perfluorocarbon, a hydrocarbon, and mixtures thereof.

28. The formulation of claim 26, wherein the propellant is selected from the group consisting of dichlorotetrafluoroethane, trichloromonofluoromethane, dichlorodifluoromethane, chloropentafluoroethane, monochlorodifluoromethane, monochlorodifluoroethane, difluoroethane, CHF₂CHF₂, 1,1,1,2-tetrafluoroethane, 1,1,2,3,3,3-heptafluoropropane, CF₃CF₃, CF₃CF₂CF₃, octafluorocyclobutane, propane, isobutane, n-butane, dimethyl ether, and mixtures thereof.

29. The formulation of claim 28, wherein the propellant is selected from the group consisting of difluoroethane, CHF₂CHF₂, 1,1,1,2-tetrafluoroethane, 1,1,1,2,3,3,3-heptafluoropropane, CF₃CF₃, CF₃CF₂CF₃, octafluorocyclobutane, and mixtures thereof.

30. The formulation of claim 19, in the form of a liquid.

31. The formulation of claim 30, wherein the liquid is an aqueous suspension.

32. The formulation of claim 30, wherein the broncho dilator is pirbuterol or a pharmacologically acceptable salt thereof.

33. The formulation of claim 32, wherein the bronchodilator is pirbuterol acetate or pirbuterol dihydrochloride.

34. The formulation of claim 30, wherein the bronchodilator is levalbuterol or a pharmacologically acceptable salt thereof.

35. The formulation of claim 34, wherein the bronchodilator is levalbuterol sulfate or levalbuterol hydrochloride.

36. The formulation of claim 30, wherein the corticosteroid is mometasone furoate monohydrate.

37. The formulation of claim 30, wherein the carrier is a sodium chloride solution.

38. The formulation of claim 1, in a unit dosage form.

39. The formulation of claim 38, wherein the unit dosage form is a hydroxypropyl methylcellulose capsule.

40. The formulation of claim 38, wherein the unit dosage form is a unit dose vial.

41. The formulation of claim 38, wherein the therapeutically effective amount of the bronchodilator is in the range of about 1 μg to about 1500 μg.

42. The formulation of claim 38, wherein the bronchodilator is pirbuterol acetate or pirbuterol dihydrochloride.

43. The formulation of claim 42, wherein the therapeutically effective amount of the bronchodilator is in the range of about 2.5 μg to about 350 μg.

44. The formulation of claim 38, wherein the bronchodilator is levalbuterol sulfate.

45. The formulation of claim 44, wherein the therapeutically effective amount of the bronchodilator is in the range of about 5.0 μg to about 150 μg.

46. The formulation of claim 38, wherein the bronchodilator is levalbuterol hydrochloride.

47. The formulation of claim 46, wherein the therapeutically effective amount of the bronchodilator is in the range of about 50 μg to about 1300 μg.

48. The formulation of claim 38, wherein the corticosteroid is anhydrous mometasone furoate or mometasone furoate monohydrate.

49. The formulation of claim 48, wherein the therapeutically effective amount of the corticosteroid is in the range of about 1 μg to about 1500 μg.

50. A pharmaceutical formulation for pulmonary drug administration, comprising:
a therapeutically effective amount of a bronchodilator selected from the group consisting of levalbuterol sulfate, pirbuterol acetate and pirbuterol dihydrochloride;
a therapeutically effective amount of a corticosteroid selected from the group consisting of anhydrous mometasone furoate and mometasone furoate monohydrate; and lactose.

51. A method for treating a patient suffering from a condition, disease or disorder that is responsive to treatment with a bronchodilator/corticosteroid combination, comprising administering to the patient, via inhalation, a pharmaceutical formulation for pulmonary drug administration, wherein the formulation comprises:
a therapeutically effective amount of a bronchodilator;
a therapeutically effective amount of a corticosteroid selected from the group consisting of mometasone and pharmacologically acceptable salts, esters and derivatives thereof; and
optionally, a pharmaceutically acceptable carrier suitable for pulmonary drug administration.

52. The method of claim 51, via oral inhalation.

53. The method of claim 51, via nasal inhalation.

54. The method of claim 51, wherein the composition is administered only as needed to treat the patient suffering from the condition, disease or disorder.

55. The method of claim 51, wherein the condition, disease or disorder is selected from the group consisting of asthma, exercise-induced asthma, bronchitis, bronchospasm, rhinitis and emphysema.

56. A pulmonary drug delivery device, comprising: a pharmaceutical formulation comprised of a therapeutically effective amount of a bronchodilator, a therapeutically effective amount of a corticosteroid selected from the group consisting of mometasone and pharmacologically acceptable salts, esters and derivatives thereof, and an optional pharmaceutically acceptable carrier suitable for pulmonary drug administration; and a means for housing and dispensing unit dosages of the formulation.

57. The drug delivery device of claim 56, comprising a dry powder inhaler, metered-dose inhaler, nebulizer or pump spray bottle.

58. The drug delivery device of claim 56, in the form of a dry powder inhaler.

59. A dry powder inhaler for orienting and positioning a capsule containing a pharmaceutical formulation to be administered via inhalation, comprising:
a dispensing chamber containing a capsule of a dry powder pharmaceutical formulation comprised of a therapeutically effective amount of a bronchodilator, a therapeutically effective amount of a corticosteroid selected from the group consisting of mometasone and pharmacologically acceptable salts, esters and derivatives thereof, and a pharmaceutically acceptable carrier suitable for pulmonary drug administration;
a tube for receiving the capsule to be oriented and dispensed;
a ramp surface extending substantially across the tube from one wall to an opposite wall thereof, and
an elongate dispensing passage having a diameter less than that of the tube and sized to receive the capsule only when the elongate axis of the capsule is generally parallel to the axis of the passage, the passage extending from an inlet end formed by an aperture in the ramp's surface to a dispensing outlet, the passage being adjacent to one wall of the tube such that the axis of the passage is parallel to, but radially offset from, an axis of the tube,
whereby when the inhaler is positioned with the passage below the tube and the axis of the passage is substantially vertical, a capsule located in the tube is guided by the ramp surface towards the inlet end of the passage.

60. A dosage form containing a pharmaceutical composition for pulmonary drug administration, the pharmaceutical composition comprising:
a therapeutically effective amount of a bronchodilator;
a therapeutically effective amount of a corticosteroid selected from the group consisting of mometasone and pharmacologically acceptable salts, esters and derivatives thereof; and
optionally, a pharmaceutically acceptable carrier suitable for pulmonary drug administration.

61. The dosage form of claim 60, in the form of a capsule.

62. The dosage form of claim 61, wherein the capsule is a hydroxypropyl methylcellulose capsule.

63. The dosage form of claim 60, wherein the therapeutically effective amount of the bronchodilator is in the range of about 1 μg to about 1500 μg.

64. The dosage form of claim 60, wherein the bronchodilator is pirbuterol acetate or pirbuterol dihydrochloride.

65. The dosage form of claim 64, wherein the therapeutically effective amount of the bronchodilator is in the range of about 2.5 μg to about 350 μg.

66. The dosage form of claim 60, wherein the bronchodilator is levalbuterol sulfate.

67. The dosage form of claim 66, wherein the therapeutically effective amount of the broncodilator is in the range of from about 5 μg to about 150 μg.

68. The dosage form of claim 60, wherein the bronchodilator is levalbuterol hydrochloride.

69. The dosage form of claim 68, wherein the therapeutically effective amount of the broncohodilator is in the range of about 50 μg to about 1300 μg.

70. The dosage form of claim 60, wherein the corticosteroid is anhydrous mometasone furoate or mometasone furoate monohydrate.

71. The dosage form of claim 70, wherein the therapeutically effective amount of the corticosteroid is in the range of about 1 μg to about 1500 μg.

72. The dosage form of claim 60, wherein the carrier is present.

73. The dosage form of claim 72, wherein the carrier is selected from the group consisting of fructose, galactose, glucose, lactitol, lactose, maltitol, maltose, mannitol, melezitose, myoinositol, palatinite, raffinose, stachyose, sucrose, trehalose, xylitol, hydrates thereof, and combinations of any of the foregoing.

74. The dosage form of claim 60, wherein the carrier is not present.
